(19) United States
(12) Patent Application Publication
(10) Pub. No.: US 2011/0072713 A1
(43) Pub. Date: Mar. 31, 2011

(54) Processing Lipids

(76) Inventors: Daniel Fleischer, Oakland, CA (US); Andrew Thompson, Oakland, CA (US); Marko Jukic, San Francisco, CA (US); Guido Radaelli, Oakland, CA (US); Jon Marshall, Oakland, CA (US)

(21) Appl. No.: 12/571,229

(22) Filed: Sep. 30, 2009

(51) Int. Cl. C10L 1/19; C07C 69/52; F28D 21/00

(52) U.S. Cl. 44/388; 560/205; 422/208

(57) ABSTRACT

A method for converting lipids to alkyl esters may include receiving a reactant comprising one or more lipids. In some cases, the reactant may include substantial amounts of polar lipids and/or free fatty acids. Some reactants may be derived from photosynthetic organisms, such as algae and/or diatoms. The reactant may be mixed with an alcohol and a catalyst to form a mixture. The mixture may be heated, for example, to a temperature between 50 and 350 degrees Celsius, including between 80 and 220 degrees Celsius. Pressure may be controlled to be between 1 and 200 bar, including between 10 and 100 bar. At least a portion of the reactant may be converted to one or more alkyl esters. A biofuel may include alkyl esters made from lipids according to various methods.

### BACKGROUND

1. Technical Field

The present invention relates generally to processing lipids, and more particularly converting lipids into fuels.

2. Description of Related Art

Some products are derived by chemically processing lipids. Some products are made by changing the lipids into other chemicals (e.g., alkanes, fatty acid alkyl esters, and the like). Transesterification is an example of such a process.

Transesterification reactions are often catalyzed with a base catalyst. Base catalyzed processes may be incompatible with acidic components such as free fatty acids (FFAs) and/or more polar lipids such as glycolipids and phospholipids. Many base catalyzed processes require lipid feedstocks having a quantity of acidic components (e.g., FFAs) that does not exceed a certain amount (e.g., 2%). Some processes are designed to receive a feedstock having precisely controlled properties, and may not be tolerant of variations in feedstock properties.

Many lipid feedstocks have concentrations of acidic and/or polar lipid components that are too large for subsequent processing, and so require pretreatment or refining prior to their use in various processes. In some transesterification processes to make biofuels, components such as free fatty acids and phospholipids are removed prior to transesterification. In such cases, the “fuel value” associated with the organic component of the free fatty acid or polar lipid may not be “carried through” to the esterified biofuel.

### SUMMARY OF THE INVENTION

A method for converting lipids to esters (e.g., to alkyl esters) may include receiving a reactant comprising one or more lipids. The reactant may be mixed with an alcohol and a catalyst to form a mixture. The mixture may be heated, typically to a temperature between 50 and 350 degrees Celsius, including between 80 and 220 degrees Celsius. Pressure may be controlled to be between 1 and 200 bar, including between 10 and 100 bar. At least a portion of the reactant may be converted to one or more esters.

In some cases, the reactant includes more than 2% (by mass, by volume, by carbon content, and/or another unit) of one or more free fatty acids. In some cases, the reactant includes over 10%, 30%, 60%, 90%, 95%, or even 99% free fatty acids and/or polar lipids. Certain reactants include less than 30% triglycerides. Some reactants include between 10 and 90% triglycerides. In some cases, the reactant may result from photosynthesis (e.g., by an algae, such as a Nannochloropsis, diatoms and/or Bacillariophyta, such as of the genera Navicula, Amphora, Thallasiosira, Chaetoceros, Nitzschia, Cyclotella, Skeletonema, Phaeodactylum, Achnanthes, Coscinodiscus, Cylindrotheca, Pseudo-Nitzschia, Thalassionema, Hantzschia, Cymbella, or Psammodictyon).

An alcohol may include methanol, ethanol, propanol, butanol, and/or another alcohol and/or mixtures of alcohols. In some cases, a mass ratio of alcohol to reactant and/or alcohol to lipids is less than 1000:1. In certain cases, the ratio is less than 10:1, or even less than 3:1.

An acidic catalyst may include a heterogeneous catalyst and/or a homogeneous catalyst. A sulfonated catalyst may be used. A pH of the mixture may be monitored and/or controlled.

A biofuel or a biopolymer may include one or more esters made from one or more lipids. A system may include a reactor for performing various reactions.

### BRIEF DESCRIPTION OF THE DRAWINGS

FIG. 1 illustrates a reactor, according to some embodiments.

FIG. 2 illustrates a method, according to some embodiments.

### DETAILED DESCRIPTION OF THE INVENTION

A reactant comprising lipids may be reacted with alcohols to convert the lipids to other chemicals. A lipid may be converted to a fatty acyl alkyl ester (which may be described as a fatty acid alkyl ester, according to the fatty acid from which the acyl moiety was derived). Exemplary esters include such as a fatty acid methyl esters. In some embodiments, an acid-catalyzed process may convert lipids to esters. An alkyl ester may be made from a lipid using an alcohol that provides an alkyl moiety to an ester derived from the lipid. Some reactants include triglycerides. Some reactants include free fatty acids. Some reactants include polar lipids such as glycolipids, phospholipids, and/or other lipids having hydrophilic head groups. Some processes include nonpolar lipids, such as a sterol-ester (e.g., an ester of cholesterol), or a polar lipid that has been modified to be hydrophobic (e.g., by attaching or substituting nonpolar moieties to polar moieties of the polar lipid). Certain embodiments receive a reactant having both free fatty acids and triglycerides, sometimes in combination with polar lipids.

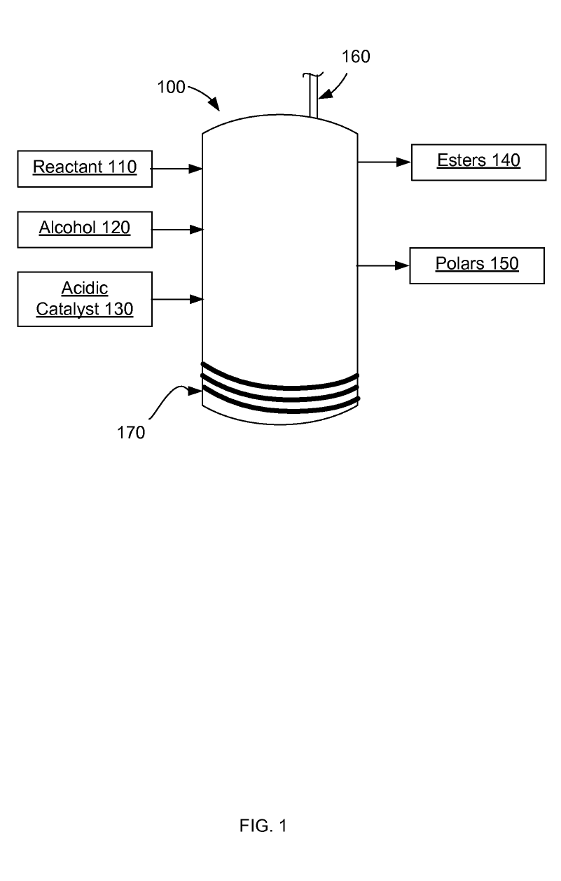

FIG. 1 illustrates a reactor, according to some embodiments. In some embodiments, reactor 100 includes a pressure vessel, and may be fabricated from stainless steel, aluminum, superalloy, and/or other materials. Reactor 100 may provide for an isobaric, isochoric, and/or isothermal process. Reactor 100 may be lined with a liner (e.g., a polytetrafluoroethylene, glass, or inconel liner). Reactor 100 may include various agitation means such as a propeller, blades, wands, jets, stir bars, baffles, paddles, and the like (not shown). Agitation may include using balls (e.g., bearing balls), sand, gravel, and other media. Reactor 100 may be lined with a liner (e.g., a polymer liner) to prevent reaction between walls of reactor 100 and substances within.

Reactor 100 may include inlets 110, 120, and 130 to receive a reactant, an alcohol, and a catalyst (respectively). Reactor 100 may include an outlet, and may include an outlet 140 for substantially nonpolar products and an outlet 150 for substantially polar products. In some cases, reactor 100 may include a separation volume (not shown) that may enhance separation of products (e.g., by density). Reactor 100 may include a gas inlet 160 (and/or a gas outlet), which may be combined with appropriate valving and pressure sensing apparatus.

Reactor 100 may include a heater 170. Heater 170 may be a heating coil disposed on the outside of the reactor (as shown in FIG. 1). Heater 170 may be disposed inside reactor 100 (e.g., immersed in the species contained within reactor 100). Infrared, radiant, microwave, inductive, and/or other heating apparatus may be used. In some cases, reactor 100 is heated, and reactor 100 heats the components within. In some cases, components within reactor 100 are heated directly (e.g., by flowing current through the components).

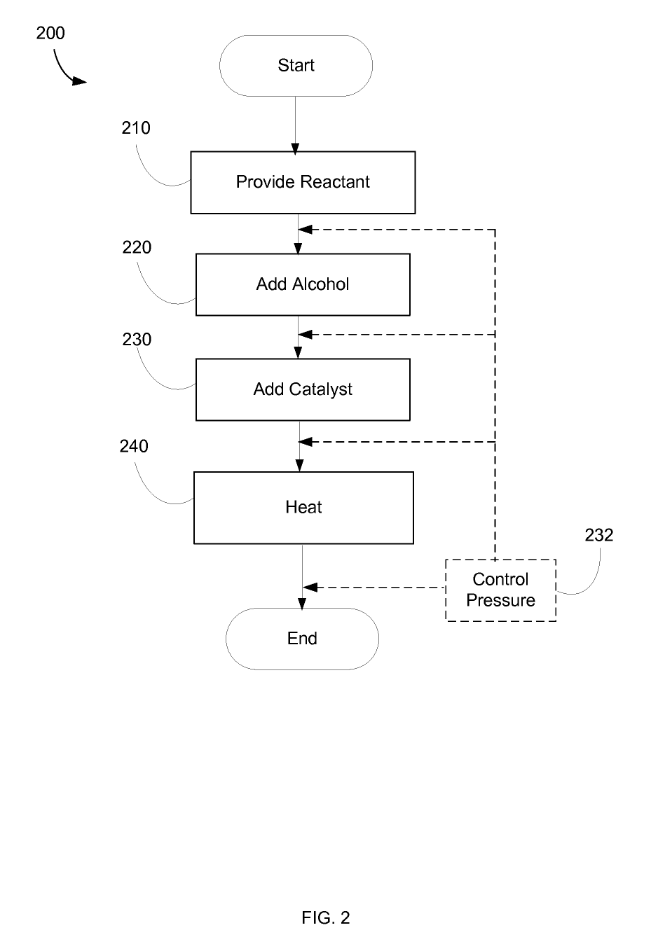

FIG. 2 illustrates a method, according to some embodiments. Method 200 may include steps 210, 220, 230, and 240, and is illustrated in FIG. 2 containing these steps for illustrative clarity. In step 210, a reactant may be provided (e.g., to a reactor such as reactor 100). In step 220, an alcohol may be added. In step 230, a catalyst (e.g., an acidic catalyst) may be added.

In some cases, a catalyst may be combined with an alcohol, and the alcohol and catalyst may be combined with the reactant. In some cases, a catalyst may be combined with the reactant, and the alcohol may be combined with the reactant and catalyst. In some cases a reactant and catalyst may be combined, and the alcohol may be combined with the reactant and catalyst. In some cases, the reactant, alcohol, and catalyst are combined substantially simultaneously.

In step 240, the mixture (e.g., of reactant, catalyst, and alcohol) may be heated. In some cases, the mixture may be heated under a controlled atmosphere. In some cases, pressure may be controlled. For example, a mixture in reactor 100 may be heated under closed conditions, such that pressure increases (e.g., via the ideal gas law). Optional step 232 illustrates control of pressure. Pressure may be controlled in conjunction with one or more steps. In some cases, pressure may be monitored (e.g., as a pressure increase as a component volatilizes). Temperature may be used to control pressure of a sealed reactor and/or vessel.

A reactant may include one or more lipids. A reactant may include glycerides (e.g., triglycerides and/or triacylglycerides). A reactant may include polar lipids such as free fatty acids, glycolipids, phospholipids, glycerophospholipids, and the like. A reactant may include lipids synthesized by algae and/or diatoms. A reactant may include one or more lipids resulting from prior chemical processes, such as waste grease, suet, tallow, yellow grease, lard, trap grease, rendered fats, ghee, used vegetable oil, and the like. A reactant may contain substantial amounts of acidic components, such as acidic lipids. A reactant may have over 5%, 10%, 20%, 40%, 60%, 90%, or even over 99% acidic components. A reactant may have between 2 and 70% acidic components.

A reactant may include triglycerides and fatty acids, and may include more than 2%, 5%, 10%, or even 40% fatty acids. A reactant may include glycolipids, phospholipids, and/or other polar lipids, and in some cases may include up to 80% polar lipids. In some embodiments, a reactant includes between 10 and 90% triglycerides and between 5 and 50% free fatty acids.

A reactant may be processed in a batch reactor and/or a continuous reactor. A reactor may receive the reactant, and may generally provide for controlled temperature, stirring and/or other agitation, and have an inlet and an outlet. A reactor may have a controlled atmosphere. A reactor may include a pressure vessel, and may provide for controlling pressure above a reaction (e.g., a pressure above liquid components). A reactant may be processed in a plurality of reactors. For example, a reactant may be partially processed in a first reactor then passed to one or more second reactors.

A reactor may receive a reactant and an alcohol. An alcohol may include methanol, ethanol, a propanol (e.g., isopropanol), a butanol, and/or other alcohols. Often, a choice of alcohol is made based on a desired alkyl moiety (e.g., the organic moiety of the alcohol) to be transferred to the lipid. Alcohol may be added in an amount that exceeds the amount of reactant and/or the amount of lipids. In some embodiments, a ratio of alcohol to reactant may be up to 10,000:1. A mass ratio of alcohol to reactant may be less than 1000:1, less than 10:1, or even less than 3:1. In some cases, an amount of alcohol may be approximately equal to an amount of reactant and/or amount of lipids.

The reactor may receive a catalyst, such as an acidic catalyst, and may mix the reactant, alcohol, and catalyst to form a mixture. An acidic catalyst may be a Bronsted acid, and may catalyze a reaction between the reactant and the alcohol.

An acidic catalyst may include a heterogeneous acid catalyst such as sulfonated graphene, sulfonated graphite, sulfonated activated carbon, sulphonated carbon nanotubes, sulfonated fullerenes, ferric sulfate, and/or sulfonated charcoal. An acidic catalyst may include graphene, graphite, activated carbon, carbon nanotubes, fullerenes and/or charcoal, which may be derivitized with a strongly acidic site. An acidic catalyst may include sulfated zirconia, sulfated tin oxide and/or a mesoporous sulfated metal oxide. An acidic catalyst may include a ceramic, metal oxide and/or metal derivitized with an active acidic site. An acidic catalyst may include a polymer having acidic sites, such as sulfonated tetrafluoroethylene, a proton exchange resin, or a polymer derivitized with a strong acidic site. A catalyst may include a solid carborane derived superacid, an acidic alumina, zeolite and/or other porous ceramic and/or siliceous material having acidic active sites.

An acidic catalyst may include a homogeneous catalyst. A homogeneous catalyst may include an acetyl chloride, an acyl halide, a strong mineral acid such as sulfuric acid, hydrochloric acid, phosphoric acid, and/or nitric acid. An acidic catalyst may include an arylsulfonic acid, a trifluoroacetic acid, a perchloric acid, a halogenated carborane superacid, a trichloroacetic acid, a trifluoromethanesulfonic acid, and the like. An acidic catalyst may include a plurality of acidic catalysts.

An acidic catalyst may be a carboxylic acid. An acidic catalyst may be carbonic acid. In some cases, a carbonic acid may be prepared by dissolving a carbonate or even carbon dioxide in an aqueous liquid. In certain cases, the carbonate or carbon dioxide may result from combustion of a carbonaceous fuel source.

An acidic catalyst may be added in an amount that depends upon the activity of the catalyst with respect to a particular reactant and alcohol. In some embodiments, a mass of acidic catalyst may be between 0.1 and 10% of the mass of the alcohol; the mass may be between 1% and 5% of the mass of the alcohol. A mass of catalyst may be between 10% and 200% of the amount of alcohol, and may be between 50% and 100% of the amount of alcohol.

A cosolvent may be added in one or more steps. A cosolvent may make a lipid/alcohol mixture monophasic, which may enhance reaction rates. In some cases, a cosolvent may improve a separation of a first phase (e.g., esters) from a second phase (e.g., a polar phase and/or a phase including polar head groups). Exemplary cosolvents may include one or more alkanes, such as hexane, pentane, and the like. A cosolvent may include an ether, such as dimethyl ether. A cosolvent may include tetrahydrofuran (THF). A cosolvent may include an ester, such as methyl acetate. A cosolvent may include a plurality of methyl esters (e.g., an alkyl ester, a plurality of alkyl esters, or even a biodiesel). A cosolvent may include a halogenated hydrocarbon such as dichloromethane, an alkene, an alkyne, a terpene, and/or a combination thereof. A cosolvent may include a combination of moieties or functionalities offering solvency, such as 2-chloro-1,1,2,-trifluoroethyl-difluoromethyl ether. A cosolvent may include a mixture of species (e.g. a 1:1 mixture of hexane: THF).

A cosolvent may be added in an amount between 0 and 0.5 times the amount of an alcohol used with a reactant. A cosolvent may be added in an amount approximately equal to an amount of alcohol (e.g., between 0.8 and 1.2 times). A cosolvent may be added in an amount greater than the amount of alcohol, more than twice the amount of alcohol, or even more than five times the amount of alcohol. In some embodiments, a cosolvent may be recovered with excess alcohol, and may be chosen to have a similar boiling point as the alcohol (if they are to be recovered together, e.g. methanol and THF). A cosolvent may be chosen to have a different boiling point than the alcohol (e.g. butanol and dimethyl ether). In some cases, a different boiling point may be used to separate the cosolvent from the alcohol (e.g., using distillation).

An atmosphere above the mixture may be controlled. A predominantly nitrogen atmosphere may be used. A predominantly argon atmosphere may be used. Air may be used. Oxygen may be used. An organic vapor (e.g., an alcohol vapor such as a Methanol vapor) may be used. An atmosphere comprising carbon dioxide (CO2) may be used. An atmosphere comprising an exhaust gas from a combustion process may be used.

Temperature of the mixture may be controlled. In some cases, the mixture may be brought to a temperature between 40 and 350 degrees Celsius. For some processes, the temperature may be brought to between 100 and 200 degrees Celsius.

The choices of temperature and amounts of catalyst, alcohol, and reactant may be interrelated. In some cases, an embodied energy associated with each of the various components (e.g., reactant, alcohol, and catalyst) may be compared to an energy associated with processing (e.g., a time and temperature). In some cases, a total energy associated with the ingredients and process may be minimized.

Pressure within the reactor may be controlled. In some cases, pressure is actively controlled (e.g., with a gas inlet and/or gas outlet). In some cases, temperature is raised above the boiling point of a component of the mixture (e.g., an alcohol) at atmospheric pressure, and the reactor is sealed, such that the pressure inside the reactor is largely dependent upon the vapor pressure of the volatile species (e.g., pressure is substantially controlled via temperature control). In some cases, pressure is determined by the amount of volatile species and the gaseous volume of the reactor. In some embodiments, temperature is increased until a certain pressure is reached.

Some embodiments include sensors to sense various parameters (e.g., clarity, pH, temperature, pressure, mass, dielectric constant, composition, molecular weight, viscosity, corrosivity, and/or other characteristics). Apparatus may monitor various sensors, and systems (mass flow, temperature, and the like) may be actuated by automated controls (solenoid, pneumatic, piezoelectric, and the like).

Some embodiments include a computer readable storage medium coupled to a processor and memory. Executable instructions stored on the computer readable storage medium may be executed by the processor to perform various methods described herein. Sensors and actuators may be coupled to the processor, providing input and receiving instructions associated with various methods. Certain instructions provide for closed-loop control of various parameters via coupled sensors providing input and coupled actuators receiving instructions to adjust parameters.

An acidic catalyst may activate a reaction between at least a portion of the reactant and at least a portion of the alcohol. In some embodiments, a conversion reaction (e.g., transesterification) provides for exchanging the organic group of the alcohol with an organic group attached to a lipid (e.g., a methyl group from methanol may be exchanged with a glycerine group attached to a lipid as a triglyceride). For example, a triglyceride may combine with three methanols to form three fatty acid methyl esters and a glycerol.

In some embodiments, acidic components (free fatty acids, polar lipids, and the like) of the reactant combine with the alcohol. For example, a free fatty acid may combine with the ethyl group of an ethanol to form a fatty acid ethyl ester. Glycolipids and/or phospholipids may combine with the alcohol. For some reactants, a polar headgroup attached to a lipid reactant may be replaced with an organic group from an alcohol.

In some cases, reactant properties (e.g., fatty acid concentration, triglyceride concentration, polar lipid concentration, and the like) are measured, and various parameters are adjusted in response to changing properties. For example, an amount of alcohol, an amount of catalyst, a temperature, and/or a dwell time may be adjusted in response to a reactant having 80% polar lipids, 10% free fatty acids, and 10% triglycerides changing to a composition of 20% polar lipids, 70% triglycerides, and 10% free fatty acids. A lookup table and/or formula may be used to adjust parameters in response to changing reactant properties.

In some embodiments, residence time (in a reactor) may be shortened when a concentration of free fatty acids increases. In some embodiments, residence time may be increased when a concentration of polar lipids increases. An amount of solvent and/or cosolvent may be adjusted according to input composition. For example, an increase in polar lipid concentration may result in a decrease in an amount of cosolvent, and/or an increase in an amount of triglycerides may result in an increase in an amount of cosolvent.

Certain embodiments include substances. A biopolymer may be fabricated by polymerizing esters derived from lipids. A biofuel may include esters converted from lipids according to various processes. In some cases, an ester profile may characterize at least a portion of a substance (e.g., a biofuel, a polymer, a chemical, and the like). An ester profile may include a histogram of quantities of various acyl groups, number of carbons, Δ^{x}, C:D, n-x, and the like. An ester profile may include a number of double bonds in one or more acyl, (or alkanoyl) chains. An ester profile may result from an associated lipid profile (e.g., a fatty acid profile) of the reactant. For example, a number of double bonds in an acyl chain may be associated with the number of double bonds in a fatty acid chain from which the alkyl-ester was derived. In some cases, the ester profile results from a combination of triglycerides and fatty acids in the reactant. In some cases, the ester profile results from a combination of triglycerides, fatty acids, and polar lipids in the reactant. In certain cases, the ester profile indicates a reactant synthesized by algae, such as Nannochloropsis, or a diatom.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

What is claimed is:

1. A method for converting lipids to alkyl esters, the method comprising:
receiving a reactant comprising one or more lipids;
mixing the reactant with an alcohol to form a mixture;
adding an acidic catalyst to the mixture;
heating the mixture to a temperature between 50 and 350 Celsius;
controlling a pressure above the heated mixture to be between 1 and 200 bar; and
converting at least a portion of the reactant to one or more alkyl esters.

2. The method of claim 1, wherein the reactant includes more than 2% of one or more free fatty acids or polar lipids.

3. The method of claim 2, wherein the reactant includes more than 10% of one or more free fatty acids or polar lipids.

4. The method of claim 1, wherein the reactant includes less than 30% of one or more triglycerides.

5. The method of claim 1, wherein the reactant includes between 10% and 90% of one or more triglycerides.

6. The method of claim 1, wherein the lipids have been synthesized by one or more algae.

7. The method of claim 6, wherein the algae include a member of the genus Nannochloropsis.

8. The method of claim 1, wherein the alcohol includes methanol, and the alkyl esters include a methyl ester.

9. The method of claim 1, wherein the alcohol includes any of ethanol, a butanol, and a propanol.

10. The method of claim 1, wherein a ratio of the alcohol to the reactant is less than 1000:1.

11. The method of claim 10, wherein the ratio is less than 10:1.

12. The method of claim 10, wherein the ratio is less than 3:1.

13. The method of claim 10, wherein the ratio is greater than 1:1.

14. The method of claim 1, wherein the acidic catalyst includes a heterogeneous catalyst.

15. The method of claim 1, wherein the acidic catalyst includes a homogeneous catalyst.

16. The method of claim 1, wherein the acidic catalyst includes a sulfonated catalyst.

17. The method of claim 1, wherein the acidic catalyst includes sulfuric acid.

18. The method of claim 1, wherein the acidic catalyst includes aqueous carbonic acid.

19. The method of claim 18, wherein the carbonic acid results from a dissolution of carbon dioxide in a liquid.

20. The method of claim 19, wherein the carbon dioxide results from combustion of fuel.

21. The method of claim 1, wherein a ratio of the acidic catalyst to the alcohol is between 0.1 and 100%.

22. The method of claim 21, wherein the ratio is between 1 and 5%.

23. The method of claim 1, wherein the temperature is between 80 and 220 Celsius.

24. The method of claim 1, wherein the pressure is between 10 and 100 bar.

25. A method for converting lipids to alkyl esters, the method comprising:
receiving a reactant comprising:
between 2 and 96% triglycerides,
between 2 and 96% free fatty acids, and
between 2 and 80% polar lipids;
mixing the reactant with an alcohol to form a mixture;
adding an acidic catalyst to the mixture;
heating the mixture to a temperature between 100 and 200 Celsius;
controlling a pressure above the heated mixture to be between 10 and 100 bar; and
converting at least a portion of the reactant to one or more alkyl esters.

26. The method of claim 25, further comprising extracting at least a portion of the alkyl esters with a nonpolar solvent.

27. A biofuel comprising esters made according to a method comprising:
receiving a reactant comprising free fatty acids and triglycerides;
mixing the reactant with an alcohol to form a mixture;
adding an acidic catalyst to the mixture;
heating the mixture to a temperature between 50 and 350 Celsius;
controlling a pressure above the heated mixture to be between 1 and 200 bar; and
converting at least a portion of the reactant to alkyl esters;
the biofuel having an ester profile indicative of the free fatty acids and the triglycerides.

28. The biofuel of claim 27, wherein at least a portion of the ester profile is indicative of a reactant comprising free fatty acids associated with any of a grease, tallow, lard, used vegetable oil, and rendered poultry fat.

29. A biofuel comprising a plurality of esters made according to a method comprising:
receiving a reactant comprising lipids from one or more algae;
mixing the reactant with an alcohol to form a mixture;
adding an acidic catalyst to the mixture;
heating the mixture to a temperature between 50 and 350 Celsius;
controlling a pressure above the heated mixture to be between 1 and 200 bar; and
converting at least a portion of the reactant to one or more alkyl esters;
the biofuel including an ester profile indicative a lipid profile associated with the lipids from the one or more algae.

30. The biofuel of claim 29, wherein the algae include any of the genus Nannochloropsis.

31. The biofuel of claim 29, wherein at least a portion of the ester profile is indicative one or more polar lipids.

32. A reactor for converting lipids to esters, the reactor comprising:
one or more inlets to receive any of a reactant, and alcohol, and an acidic catalyst into a sealed volume;
mixing means to mix the reactant, alcohol, and catalyst; and
a heater to heat the mixture.

33. The reactor of claim 32, further comprising a pressure control apparatus configured to monitor a pressure inside the volume.

34. The reactor of claim 33, wherein the pressure control apparatus adjusts the pressure in response to the monitored pressure.
